(19) United States
(12) Patent Application Publication
(10) Pub. No.: US 2025/0268612 A1
(43) Pub. Date: Aug. 28, 2025

(54) CATHETERS, DEVICES, AND METHODS FOR REMOVAL OF MATERIAL FROM HOLLOW BODIES

(71) Applicant: BARD PERIPHERAL VASCULAR, INC., Franklin Lakes, NJ (US)

(72) Inventors: Matt CASIRARO, Tempe, AZ (US); Matthew RILEY, Scottsdale, AZ (US)

(73) Assignee: BARD PERIPHERAL VASCULAR, INC., Franklin Lakes, NJ (US)

(21) Appl. No.: 18/858,017

(22) PCT Filed: Apr. 27, 2022
(86) PCT No.: PCT/US2022/026503

§ 371 Date: Oct. 18, 2024

(51) Int. Cl. A61B 17/22; A61B 17/00; A61B 17/3207

(52) U.S. Cl. CPC A61B 17/22; A61B 17/00234; A61B 17/320758; A61B 2017/00238; A61B 2017/00305; A61B 2017/00398; A61B 2017/22079; A61B 2217/005

(57) ABSTRACT

A catheter assembly for removal of material from hollow bodies may include an outer sheath, and an inner thrombectomy catheter disposed inside of the outer sheath. The inner thrombectomy catheter may include a distal end and a conveyer. The distal end may include a distal tip being closed and a lateral opening such that the lateral opening receives a removed material. The conveyer may convey the removed material toward a proximal end of the inner thrombectomy catheter. An expandable member may be disposed between the outer sheath and the inner thrombectomy catheter. The expandable member may expand radially when deployed from the outer sheath and contract when retracted in the outer sheath.

### TECHNICAL FIELD

The present disclosure relates to catheters, devices, and methods for removal of material from hollow bodies (e.g., blood vessels), and more particularly catheters, devices, and methods including an expandable member surrounding an inner thrombectomy catheter.

### BACKGROUND

Deposits, for example in the form of thrombi and emboli in blood vessels, reduce the sectional area of flow of hollow bodies, such as blood vessels, arteries and veins. This can lead to congestion or to a total blockage (emboli) of the blood vessel. The health consequences may be circulatory disorders or, in extreme cases, the failure of extremities or even vital organs. The removal of thrombi may be relatively complex, since the removed deposits consist of larger and smaller fragments which tend to stick together, but cannot be comminuted, and therefore the particles remaining in the blood can float in the blood. This leads to the formation of clumps and to blocking of the vein system. It may therefore be useful to have devices providing reliable removal of thrombi from blood vessels or solid materials from any other hollow bodies, reducing chances of parts of solid materials being distributed over the working area.

### SUMMARY

To reduce chances of parts of solid bodies being distributed over the working area in blood vessels or other hollow bodies, a thrombectomy catheter may be used for the removal. Embodiments of the present disclosure are directed to catheters, devices, and methods for removal of material from hollow bodies with an expandable member surrounding an inner thrombectomy catheter as will be described in greater detail below.

In one embodiment, a catheter assembly for removal of material from hollow bodies may include an outer sheath, and an inner thrombectomy catheter disposed inside of the outer sheath. The inner thrombectomy catheter may include a distal end and a conveyer, the distal end having a distal tip being closed and a lateral opening such that the lateral opening receives a removed material, and the conveyer conveying the removed material toward a proximal end of the inner thrombectomy catheter. The catheter assembly may include an expandable member disposed between the outer sheath and the inner thrombectomy catheter expanding radially when deployed from the outer sheath and contracting when retracted in the outer sheath.

In another embodiment, a method of using a catheter assembly for removal of material from hollow bodies may include positioning the catheter assembly at a removal site. The catheter assembly may include an outer sheath, and an inner thrombectomy catheter disposed inside of the outer sheath. The inner thrombectomy catheter may include a distal end and a conveyer, the distal end having a distal tip being closed and a lateral opening such that the lateral opening receives a removed material, and the conveyer conveying the removed material toward a proximal end of the inner thrombectomy catheter. The catheter assembly may include an expandable member disposed between the outer sheath and the inner thrombectomy catheter expanding radially when deployed from the outer sheath and contracting when retracted in the outer sheath. The method may include advancing the expandable member in a distal direction of the inner thrombectomy catheter such that the expandable member is deployed and expanded radially.

In yet another embodiment, a surgical device includes a catheter assembly for removal of material from hollow bodies. The catheter assembly may include an outer sheath, an inner thrombectomy catheter disposed inside of the outer sheath. The inner thrombectomy catheter may include a distal end and a conveyer. The distal end may have a distal tip and a lateral opening such that the lateral opening receives a removed material. The conveyer may convey the removed material toward a proximal end of the inner thrombectomy catheter. An expandable member may be disposed between the outer sheath and the inner thrombectomy catheter expanding radially when deployed from the outer sheath and contracting when retracted in the outer sheath. The surgical device may include a guidewire extendable through the inner thrombectomy catheter.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 schematically depicts one embodiment of a catheter assembly for removal of material from hollow bodies, wherein an expandable member is retracted in an outer sheath, according to one or more embodiments shown and described herein;

FIG. 2 schematically depicts the catheter assembly of FIG. 1, wherein the expandable member is expanded, according to one or more embodiments shown and described herein;

FIG. 3A schematically depicts a cross sectional view of the catheter assembly of FIG. 2 taken along section line 3A-3A, according to one or more embodiments shown and described herein;

FIG. 3B schematically depicts a cross sectional view of an inner thrombectomy catheter of FIG. 2 taken along section line 3B-3B, according to one or more embodiments shown and described herein;

FIG. 4 schematically depicts another embodiment of the catheter assembly wherein the expandable member having one prong, which is expanded, according to one or more embodiments shown and described herein; and

FIG. 5 schematically depicts the catheter assembly of FIGS. 1 to 3B advanced through a hollow body, according to one or more embodiments shown and described herein.

Reference will now be made in greater detail to various embodiments of the present disclosure, some embodiments of which are illustrated in the accompanying drawings. Whenever possible, the same reference numerals will be used throughout the drawings to refer to the same or similar parts.

### DETAILED DESCRIPTION

Embodiments described herein are directed to catheters, devices, and methods for removal of material from hollow bodies such as, but not limited to thrombectomy, embolectomy, etc. For example, a catheter may be placed in a blood vessel or in an organ to remove solid bodies such as blood clots or foreign materials therefrom. When solid bodies are removed, smaller fragments may remain at the removal location. Embodiments of the present disclosure may provide improved removal of solid bodies. For example, in some embodiments, a catheter may have an expandable member surrounding an inner thrombectomy catheter for improved removal of the solid bodies. These and additional features and benefits will be described in greater detail herein.

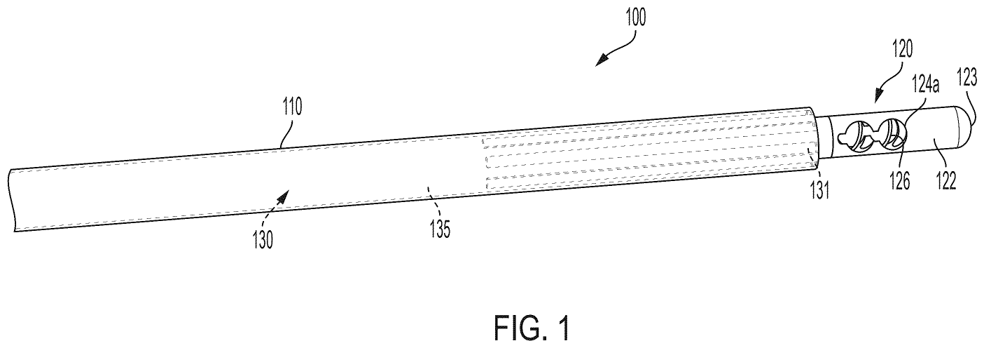

Referring now to FIG. 1, a catheter assembly 100 for removing material from hollow bodies, is schematically depicted. The catheter assembly 100 may include an outer sheath 110, an inner thrombectomy catheter 120, and an expandable member 130.

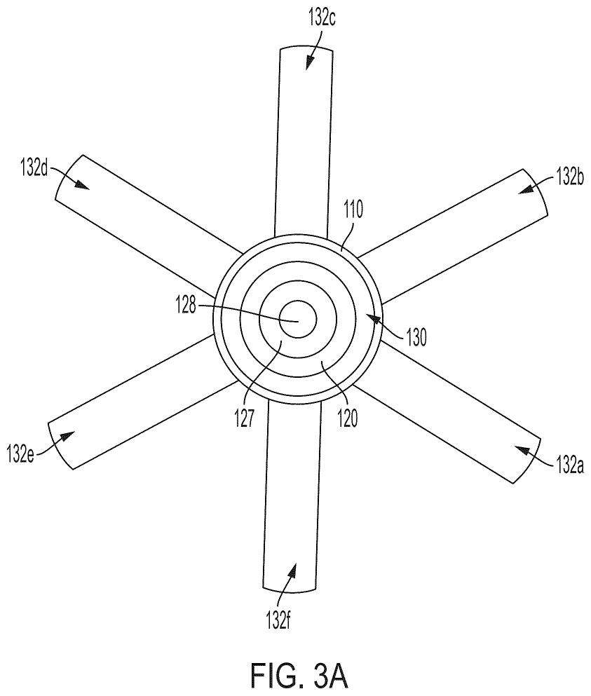

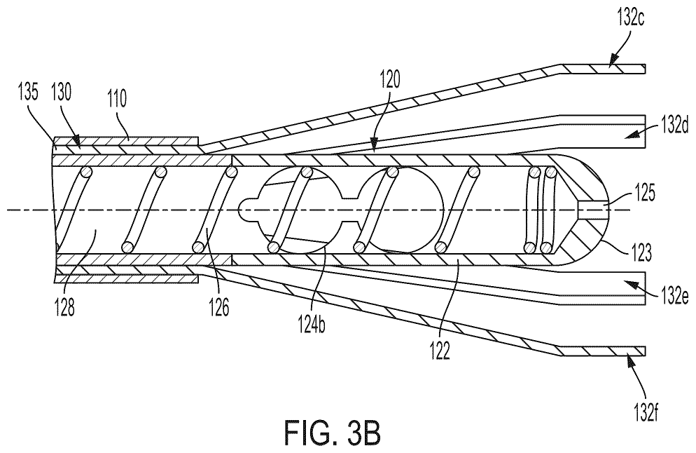

The inner thrombectomy catheter 120 may be disposed inside of and extend through the outer sheath 110 and the expandable member 130. The inner thrombectomy catheter 120 may have a distal end 122, a lateral opening 124a, and a conveyer 126. The distal end 122 may have a distal tip 123 being closed such that materials may not be received through the distal tip 123. The closed configuration of the distal tip 123 may allow the lateral openings 124a and 124b (shown in FIG. 3B) to receive materials. The closed distal tip 123 may have a lumen 125 (shown in FIG. 3B) in which a guidewire may pass through to guide the catheter assembly 100 through hollow bodies. The conveyer 126 may convey the removed material received through the lateral openings 124a and 124b toward a proximal end of the inner thrombectomy catheter 120. Further details of the inner thrombectomy catheter 120 will be discussed with respect to FIG. 3B.

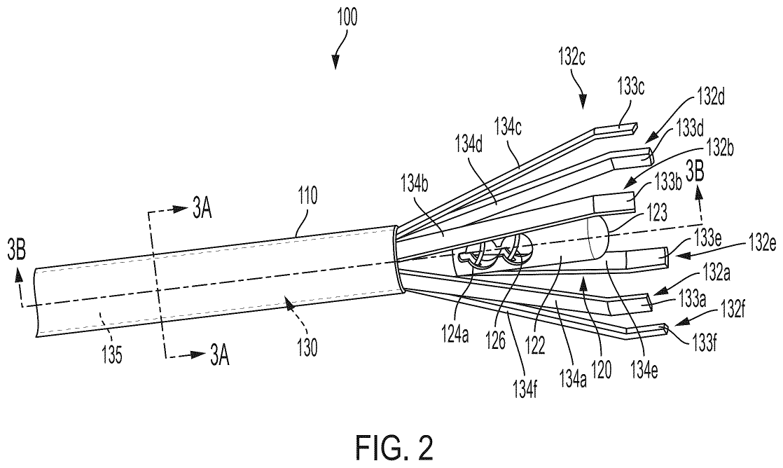

Still referring to FIG. 1, the expandable member 130 may be disposed between the outer sheath 110 and the inner thrombectomy catheter 120. The expandable member 130 is hidden under the outer sheath 110 and shown as dashed lines. The expandable member 130 may include at least one prong that extends axially in a distal direction of the inner thrombectomy catheter 120. In some embodiments, the expandable member 130 may be substantially symmetric around an axis of the inner thrombectomy catheter 120. For example, there are six prongs in total, three prongs 132a, 132b, and 132c shown as a dashed line and three prongs 132d, 132e, and 132f (FIG. 2) on the opposite side of the catheter assembly 100. The prongs 132a-132f may be disposed radially around the inner thrombectomy catheter 120. The 132a-132f may be arranged with substantially the same interval in a radial direction, and the width and the length may be substantially the same.

The expandable member 130 is illustrated in a retracted position in FIG. 1. Referring also to FIG. 2, the expandable member 130 may be movable or slidable along the inner thrombectomy catheter 120 in the longitudinal direction relative to the inner thrombectomy catheter 120 to move between the retracted position and a deployed position. The expandable member 130 is contracted when in retracted in the outer sheath 110. When in the retracted position, a distal end 131 of the expandable member 130 may be covered by the outer sheath 110. In other words, in the retracted position, the expandable member 130 may terminate at the distal end 131 located inside the outer sheath 110. The prongs 132a-132f may be contracted in the outer sheath 110 when the expandable member 130 is retracted into the outer sheath 110. In some embodiments, the distal end 131 of the expandable member 130 may be partially covered by the outer sheath 110 when in the retracted position such that the prongs 132a-132f are partially exposed but still contracted. The prongs 132a-132f may stay substantially parallel to the axis of the inner thrombectomy catheter 120 when the expandable member 130 is in the retracted position.

The outer sheath 110 may obstruct expansion of the prongs 132a-132f when the prongs 132a-132f are at least partially covered by the outer sheath 110. The outer sheath 110 may press down the prongs 132a-132f to obstruct the expansion. The outer sheath 110 may be made out of materials include a polymer material (e.g., polypropylene, polyester, or the like) or any other materials that may have strength to withstand the expanding forces of the expandable member 130 when the prongs 132a-132f are housed in the outer sheath 110.

Referring to FIG. 2, the expandable member 130 is in the deployed position. The prongs 132a-132f may have a tendency of expanding radially, in other words, a self-expanding mechanism. Therefore, the prongs 132a-132f may spread out to form a funnel or a basket like shape which may be narrowed or tapered radially inward toward the proximal end of the inner thrombectomy catheter 120. In some embodiments, the expandable member 130 may be made out of materials having a tendency of expanding radially. Non-limiting examples of materials suitable for forming the expandable member 130 include a shape-memory material (e.g., nitinol, stainless steel, or the like) and may be pre-curved to expand radially outward as shown when deployed. In some embodiments, only the prongs 132a-132f may be made out of the material having the tendency of expanding radially. The prongs 132a-132f may be cut out of a tube that forms the expandable member 130. Alternatively, the prongs 132a-132f may be formed separately from the tube portion of the expandable member 130 and then connected together by any suitable method.

As explained above, the expandable member 130 is radially expanded when deployed from the outer sheath 110 and released from radial compression by the outer sheath 110. When in the deployed position, the prongs 132a-132f may no longer be covered by the outer sheath 110 and expand radially such forming an expanded shape that surrounds the distal end 122 of the inner thrombectomy catheter 120. The outermost diameter of the expanded shape of the expandable member 130 may be larger than the outermost outer diameter of the outer sheath 110. The prongs 132a-132f may gradually expand while the expandable member 130, moving away from one another in both the radial and circumferential directions as the expandable member 130 is moved from the retracted position to the deployed position. The prongs 132a-132f may be fully expanded when the expandable member 130 is moved to the deployed position and the prongs 132a-132f are not covered by the outer sheath 110. Each of the prongs 132a-132f may have respective proximal portions 134a-134f and respective distal portions 133a-133f that extend outward from respective the proximal portions 134a-134f at an oblique angle. When the prongs 132a-132f are fully expanded, distal portions 133a-133f of the prongs 132a-132f are substantially parallel to the axis of the inner thrombectomy catheter 120. For example, the distal portions 133a-133f of the prongs 132a-132f are angled inwardly in the radial direction to be substantially parallel to the axis. An edge of a distal portions 133a-133f of each of the prongs 132a-132f may be flat as shown in FIG. 2, or may have rounded or tapered shape.

Still referring to FIG. 2, the prongs 132a-132f may radially overlap the inner thrombectomy catheter 120 when the expandable member is deployed from the outer sheath 110. For example, the prongs 132a-132f may extend beyond the distal tip 123 in the longitudinal direction. The length of prongs 132a-132f may depend on the size of the catheter assembly 100. The length of prongs 132a-132f may be between about 1 centimeters to about 10 centimeters. The length of prongs 132a-132f may be between about 1 centimeters to about 2 centimeters. It should be noted that the prongs 132a-132f may extend at least beyond the proximal end of the lateral openings 124a and 124b in the fully deployed position to assist removal of materials from hollow bodies through the lateral opening 124a and 124b. In some embodiments, the prongs 132a-132f may have lengths such that the distal tips of the prongs 132a-132f extend distally beyond the distal tip 123 inner thrombectomy catheter 120 in the deployed position, as shown, or may have lengths such that the distal tips terminate proximal of the distal tip 123 inner thrombectomy catheter 120 in the fully deployed position.

Referring to FIG. 3A, a cross sectional view of the catheter assembly 100 in FIG. 2 taken along line 3A-3A is illustrated. The six prongs 132a-132f may be arranged substantially radially symmetrical. The prongs 132a-132f may also be arranged substantially bilaterally symmetrical. In some embodiments, the number of prongs may be one to five. The number of prongs may be more than six. The number of prongs is not limited as long as may form a funnel like shape.

FIG. 3A further illustrates a structure inside of the catheter assembly 100. From outside to inside, the outer sheath 110, the expandable member 130, the inner thrombectomy catheter 120, a conveyer support 127 that may support and rotate the conveyer 126 (FIG. 2), and a hollow body 128 of the inner thrombectomy catheter 120 may be disposed in that order. It is noted that there may be a clearance between each of the components of the catheter assembly 100 such that the components may be moved independently with respect to each other. For example, the expandable member 130 may be moved in the longitudinal direction between the retracted position and the deployed position. The expandable member 130 may also be rotated around the longitudinally extending axis. The conveyer support 127 may be rotated to rotate the conveyer 126 to convey materials through the hollow body 128 in a lateral direction. In some embodiments, the body of the inner thrombectomy catheter 120 may not be rotated and only conveyer 126 is rotatable relative to the inner thrombectomy catheter 120.

The components may be moved manually by a user of the catheter assembly 100 or may be moved by a motor that is coupled to one or more of the components. For example, the expandable member 130 may be moved and/or rotated manually by a user through a thumb gear or the like. The thumb gear may be coupled to a shaft 135 of the expandable member. The expandable member 130 may be moved and/or rotated by a motor connected to the expandable member 130 through the shaft 135. Therefore, the shaft 135 may be moved in a longitudinal direction relative to the inner catheter 120 and the outer sheath 110. In some embodiments, the same thumb gear or motor that rotates the shaft 135 of the expandable member 130 may also be coupled to the conveyer 126 and/or the conveyer support 127 to rotate the conveyer 126. In other embodiments, a separate motor or thumb gear may be utilized to rotate the conveyer 126. The expandable member 130 and the conveyer 126 may rotate or move relative to the inner thrombectomy catheter 120.

Referring to FIG. 3B, a longitudinal sectional view of the catheter in FIG. 2 taken along line 3B-3B is illustrated. A lateral opening 124b may be disposed on the other side of the lateral opening 124a (shown in FIG. 2). The lateral openings 124a and 124b may face one another and overlap when viewed from a side. The lateral openings 124a and 124b may receive materials there through to be delivered to the hollow body 128. In some embodiments, the lateral openings 124a and 124b may be formed by two circular holes arranged axially in succession in relation to the longitudinal axis of the inner thrombectomy catheter 120. The circumferences of the circular holes overlap to form the lateral openings 124a and 124b. The inner edges of the lateral openings 124a and 124b may be shearing edges to assist cutting of materials entering the lateral openings 124a and 124b. Any suitable shape can be used for the lateral openings 124a and 124b, such as any suitable regular or irregular shape.

The conveyer 126 may be rotated to convey materials toward proximal end of the inner thrombectomy catheter 120 through the hollow body 128. The conveyer 126 may be an auger or a coil that may have a spiral shape forming a type of displacement device to convey materials when rotated. The edges of the conveyer 126 may be sheared or serrated to further break down materials to small pieces. The rotation of the conveyer 126 may create aspirational forces to draw the materials to enter the lateral openings 124a and 124b. The conveyer 126 may be rotated relative to the inner thrombectomy catheter 120. In some embodiments, the inner thrombectomy catheter 120 may be coupled to a vacuum member that applies aspirational forces. The inner thrombectomy catheter 120 may have a reservoir coupled to the proximal end of the inner thrombectomy catheter 120 to collect the removed materials conveyed toward the proximal end by the conveyer 126 through the hollow body 128.

The inner thrombectomy may have the lumen 125 in the distal tip 123 to pass a guidewire through to guide the catheter assembly 100 to be positioned at a target treatment site or a removal site. The distal tip 123 with the lumen 125 is closed except for the lumen 125 to disallow materials may from passing through the distal tip 123 with the guidewire located therein. The lumen 125 may have an inner diameter that may allow the guidewire to pass through but not large enough to receive the materials removed from the removal site. In some embodiments, the inner thrombectomy catheter 120 may not have the lumen 125 and the distal tip 123 is completely closed. The guidewire may be attached to the distal tip 123 of the inner thrombectomy catheter 120.

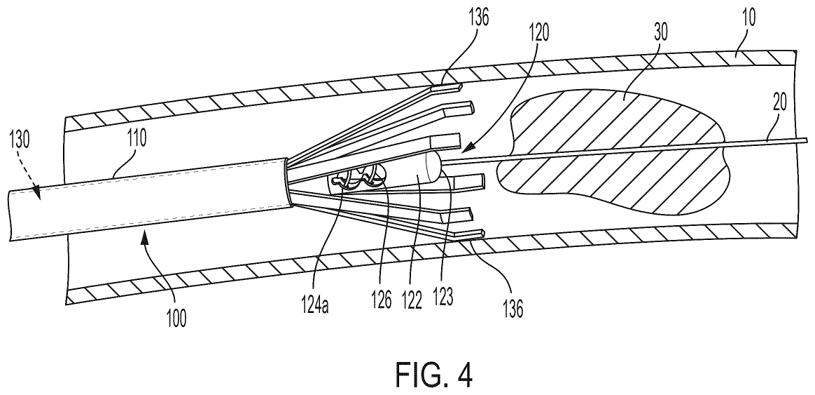

Referring to FIG. 4, the catheter assembly 100 of FIGS. 1-3B is advanced through a hollow body. The catheter assembly 100 may be inserted into the hollow body in a distal direction and the expandable member 130 may be advanced in the distal direction such that the expandable member 130 is deployed and expanded radially. For example, the catheter assembly 100 may be advanced through a blood vessel 10 and may be guided by a guidewire 20 that is inserted in the blood vessel 10 before or while inserting the catheter assembly 100 in the blood vessel 10. The guidewire 20 may be attached to the distal tip 123 or may be passed through the lumen 125 of the inner thrombectomy catheter 120. The guidewire 20 and/or the catheter assembly 100 may be flexible enough to be advanced through twisted or curved blood vessels.

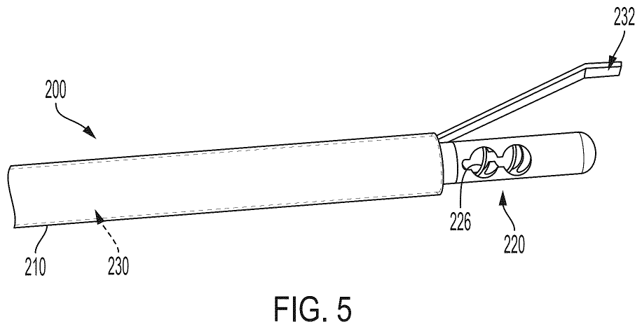

The catheter assembly 100 may be positioned at a removal site, where material to be removed from the hollow body is located. For example, the catheter assembly 100 may be advanced through the blood vessel 10 where deposits 30 (e.g., thrombi, emboli, or the like) are located. The deposits 30 may reduce the sectional area of flow of the blood vessel 10. The catheter assembly 100 may be advanced until immediately adjacent the deposits 30. Once positioned at the removal site, the expandable member 130 may be advanced in the distal direction such that the expandable member 130 is deployed and expanded radially as illustrated in FIG. 5. The expandable member 130 expands so that to form a funnel-like shape that surrounds the distal end 122 of the inner thrombectomy catheter 120.

In some embodiments, the expandable member 130 may be expanded to contact the wall of the blood vessel 10 in the fully deployed position, as illustrated in FIG. 4. In some embodiments, the expandable member 130 may press the blood vessel radially (e.g., slightly expanded to fit over), or the expandable member 130 may be expanded such that the outer most diameter of the funnel shape is smaller than the inner diameter of the blood vessel 10. The end portions of the prongs 132a-132f may be angled inwardly in the radial direction substantially parallel to the axis such that the end portions of the prongs 132a-132f may be substantially parallel to the inner surface of the blood vessel 10. In some embodiments, the prongs 132a-132f may be angled further such that the bent portion 136 of each of the prongs 132a-132f may touch the blood vessel 10. The prongs 132a-132f may scrape and cleave deposits adhered to a blood vessel wall by a lateral movement with lateral push and pull toward the blood vessel wall. Also, the prongs 132a-132f may scrape and cleave deposits adhered to a blood vessel wall by being moved back and forth in a longitudinal direction along the blood vessel wall. The angled configurations of the prongs 132a-132f may protect the blood vessel 10 from inadvertently pierced or scratched by the expandable member 130. The distal end of the prongs 132a-132f may have a shape that may protect the blood vessel 10 from inadvertently pierced or scratched by the expandable member 130. For example, the edge of each of the distal end may be flat or rounded.

In some embodiment, the catheter assembly 100 may be advanced or retreated to adjust the position while the expandable member 130 is deployed and expanded. In other embodiments, the expandable member 130 may be retracted and contracted in the outer sheath 110 to advance or retreat the catheter assembly 100.

The expandable member 130 may be rotated after expansion around an axis of the inner thrombectomy catheter 120. The rotation of the expandable member 130 may generate aspirational forces to draw the deposits 30 toward the inner thrombectomy catheter 120. The rotation of the expandable member 130 may assist separating the deposits 30 adhered to the blood vessel 10 by touching or scratching the deposits 30 with the prongs 132a-132f. The expandable member 130 may be rotatable in both clockwise and counterclockwise. Rotating the expandable member 130 in both directions may further assist removing the deposits 30 from the blood vessel 10.

The inner thrombectomy catheter 120 may apply aspiration forces through the lateral openings 124a and 124b to draw the deposits 30 toward the inner thrombectomy catheter 120. The aspiration forces may be applied by rotating the conveyer 126 and/or by activating the vacuum member coupled to the inner thrombectomy catheter 120. The rotation of the conveyer support 127 and/or the activation of the vacuum member may proceed before and/or after the expandable member 130 is deployed and expanded. When the aspirational forces are applied, the expandable member 130 may prevent the vessel wall of the blood vessel 10 from vacuumed into the lateral openings 124a and 124b when expanded. The expandable member 130 may protect the vessel wall from cut or scratch caused by the conveyer 126 and/or the edges of the lateral openings 124a and 124b which may be sheared or serrated to cut the deposits 30.

The deposits 30 may be received through the lateral openings 124a and 124b as discussed above. During the removal of the deposits 30, the deposits 30 may be fragmented to larger and/or smaller particles floating in the blood. The expandable member 130 may help catch or guide the fragments such that the fragments may be drew toward the lateral openings 124a and 124b and collected from the blood. The expandable member 130 may reduce chances of fragments being distributed in the blood vessel 10. The blood may still flow through gaps between the prongs 132a-132f, and thereby through the blood vessel 10.

After the removal of the deposits 30 from the blood vessel 10, the catheter assembly 100 may be withdrawn from the blood vessel 10. The expandable member 130 may be retracted in the outer sheath 110 such that the prongs 132a-132f are contracted in the outer sheath 110 while withdrawing the catheter assembly 100 from the blood vessel 10.

Referring to FIG. 5, another catheter assembly 200 is illustrated. The catheter assembly 200 of FIG. 4 may include an expandable member 230 with one prong 232. The expandable member 230 may be movable or slidable along an inner thrombectomy catheter 220 in a longitudinal direction relative to the inner thrombectomy catheter 220 to move between the retracted position and a deployed position. The expandable member 230 is contracted when in retracted in the outer sheath 210. The expandable member 230 may also be rotated around the longitudinally extending axis. The conveyer 126 may be rotated to convey materials in a lateral direction. In some embodiments, the body of the inner thrombectomy catheter 220 may not be rotated and only conveyer 226 is rotatable relative to the inner thrombectomy catheter 220. It should be noted that the rest of the configuration is substantially equivalent to that of the embodiments of the catheter assembly 100, other than noted differently. Therefore, the description of the substantially equivalent configuration is omitted.

A surgical device, in some embodiments, may include the catheter assembly 100, a guidewire attached to the catheter assembly 100 or configured to as a standalone component of the surgical device. The surgical device may further include a driving mechanism such as a motor to rotate the expandable member 130. The driving mechanism may also rotate the conveyer 126 of the inner thrombectomy catheter 120 or the surgical device has an additional driving mechanism to independently rotate the conveyer 126. An aspiration device may be coupled to the inner thrombectomy catheter 120 to create suction within the inner thrombectomy catheter 120. The aspiration device may have a vacuum generating member to generate the aspirational forces. It should be noted that the rest of the configuration is substantially equivalent to that of the embodiments of the catheter assembly 100, other than noted differently. Therefore, the description of the substantially equivalent configuration is omitted.

In other embodiments, the surgical device may include the catheter assembly 200, a guide wire attached to the catheter assembly 200 or configured to as a standalone component of the surgical device. The surgical device may further include a driving mechanism such as a motor to rotate the expandable member 230. The driving mechanism may also rotate the conveyer 226 of the inner thrombectomy catheter 220 or the surgical device has an additional driving mechanism to independently rotate the conveyer 226. An aspiration device may be coupled to the inner thrombectomy catheter 220 to create suction within the inner thrombectomy catheter 220. The aspiration device may have a vacuum generating member to generate the aspirational forces. It should be noted that the rest of the configuration is substantially equivalent to that of the embodiments of the catheter assembly 200, other than noted differently. Therefore, the description of the substantially equivalent configuration is omitted.

Embodiments can be described with reference to the following numerical clause:

1. A catheter assembly for removal of material from hollow bodies, comprising: an outer sheath; an inner thrombectomy catheter disposed inside of the outer sheath, the inner thrombectomy catheter including a distal end and a conveyer, the distal end having a distal tip being closed and a lateral opening such that the lateral opening receives a removed material, and the conveyer conveying the removed material toward a proximal end of the inner thrombectomy catheter; and an expandable member disposed between the outer sheath and the inner thrombectomy catheter expanding radially when deployed from the outer sheath and contracting when retracted in the outer sheath.

2. The catheter assembly of any preceding clause, wherein the expandable member includes at least one prong that extends axially in a distal direction of the inner thrombectomy catheter.

3. The catheter assembly of any preceding clause, wherein the expandable member includes a plurality of prongs that extend axially in a distal direction of the inner thrombectomy catheter.

4. The catheter assembly of any preceding clause, wherein the expandable member includes six prongs that extend axially in a distal direction of the inner thrombectomy catheter.

5. The catheter assembly of any preceding clause, wherein an end portion of the expandable member is configured to be substantially parallel to a longitudinal direction of the inner thrombectomy catheter when the expandable member is deployed and expanded.

6. The catheter assembly of any preceding clause, wherein the expandable member is substantially symmetric around an axis of the inner thrombectomy catheter.

7. The catheter assembly of any preceding clause, wherein the expandable member is rotatable around an axis of the inner thrombectomy catheter independently of the inner thrombectomy catheter.

8. The catheter assembly of any preceding clause, wherein the distal end of the inner thrombectomy catheter and the expandable member are radially overlapped when the expandable member is deployed from the outer sheath while performing removal of the material.

9. A method of using a catheter assembly for removal of material from hollow bodies, comprising: positioning the catheter assembly at a removal site, the catheter assembly comprising: an outer sheath; an inner thrombectomy catheter disposed inside of the outer sheath, the inner thrombectomy catheter including a distal end and a conveyer, the distal end having a distal tip being closed and a lateral opening such that the lateral opening receives a removed material, and the conveyer conveying the removed material toward a proximal end of the inner thrombectomy catheter, and an expandable member disposed between the outer sheath and the inner thrombectomy catheter expanding radially when deployed from the outer sheath and contracting when retracted in the outer sheath; and advancing the expandable member in a distal direction of the inner thrombectomy catheter such that the expandable member is deployed and expanded radially.

10. The method of any preceding clause, further comprising rotating the expandable member around an axis of the inner thrombectomy catheter.

11. The method of any preceding clause, further comprising applying aspiration forces through the lateral opening.

12. The method of any preceding clause, further comprising receiving the removed material through the lateral opening, and conveying the removed material toward the proximal end of the inner thrombectomy catheter by the conveyer.

13. The method of any preceding clause, further comprising retracting the expandable member in the outer sheath.

14. A surgical device, comprising: a catheter assembly for removal of material from hollow bodies, comprising: an outer sheath, an inner thrombectomy catheter disposed inside of the outer sheath, the inner thrombectomy catheter including a distal end and a conveyer, the distal end having a distal tip and a lateral opening such that the lateral opening receives a removed material, and the conveyer conveying the removed material toward a proximal end of the inner thrombectomy catheter, and an expandable member disposed between the outer sheath and the inner thrombectomy catheter expanding radially when deployed from the outer sheath and contracting when retracted in the outer sheath; and a guidewire extendable through the inner thrombectomy catheter.

15. The surgical device of any preceding clause, wherein the expandable member is rotatable around an axis of the inner thrombectomy catheter independently of the inner thrombectomy catheter.

16. The surgical device of any preceding clause, further comprising a driving mechanism to rotate the expandable member.

17. The surgical device of any preceding clause, wherein the expandable member includes at least one prong that extends axially in a distal direction of the inner thrombectomy catheter.

18. The surgical device of any preceding clause, wherein the expandable member includes a plurality of prongs that extend axially in a distal direction of the inner thrombectomy catheter.

19. The surgical device of any preceding clause, wherein the distal end of the inner thrombectomy catheter and the expandable member are radially overlapped when the expandable member is deployed from the outer sheath while performing removal of the material.

20. The surgical device of any preceding clause, further comprising an aspiration device coupled to the inner thrombectomy catheter to create suction within the inner thrombectomy catheter.

It should now be understood that embodiments of the present disclosure are directed to catheters, devices, and methods for removal of material from hollow bodies with an expandable member surrounding an inner thrombectomy catheter. In some embodiments, catheters, devices, and methods may be used to remove materials from a blood vessel. More particularly, the expandable member may be disposed between the outer sheath and the inner thrombectomy catheter and may expand radially when deployed from the outer sheath and contracting when retracted in the outer sheath. The inner thrombectomy catheter may be disposed under the expandable member and may include a distal end and a conveyer. The distal end may have a distal tip being closed and a lateral opening such that the lateral opening receives a removed material. The conveyer may convey the removed material toward a proximal end of the inner thrombectomy catheter. These and additional features and benefits are described in greater detail herein.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

What is claimed is:

1. A catheter assembly for removal of material from hollow bodies, comprising:
an outer sheath;
an inner thrombectomy catheter disposed inside of the outer sheath, the inner thrombectomy catheter including a distal end and a conveyer, the distal end having a distal tip being closed and a lateral opening such that the lateral opening receives a removed material, and the conveyer conveying the removed material toward a proximal end of the inner thrombectomy catheter; and
an expandable member disposed between the outer sheath and the inner thrombectomy catheter expanding radially when deployed from the outer sheath and contracting when retracted in the outer sheath.

2. The catheter assembly of claim 1, wherein the expandable member includes at least one prong that extends axially in a distal direction of the inner thrombectomy catheter.

3. The catheter assembly of claim 1, wherein the expandable member includes a plurality of prongs that extend axially in a distal direction of the inner thrombectomy catheter.

4. The catheter assembly of claim 1, wherein the expandable member includes six prongs that extend axially in a distal direction of the inner thrombectomy catheter.

5. The catheter assembly of claim 1, wherein an end portion of the expandable member is configured to be substantially parallel to a longitudinal direction of the inner thrombectomy catheter when the expandable member is deployed and expanded.

6. The catheter assembly of claim 1, wherein the expandable member is substantially symmetric around an axis of the inner thrombectomy catheter.

7. The catheter assembly of claim 1, wherein the expandable member is rotatable around an axis of the inner thrombectomy catheter independently of the inner thrombectomy catheter.

8. The catheter assembly of claim 1, wherein the distal end of the inner thrombectomy catheter and the expandable member are radially overlapped when the expandable member is deployed from the outer sheath while performing removal of the material.

9. A method of using a catheter assembly for removal of material from hollow bodies, comprising:
positioning the catheter assembly at a removal site, the catheter assembly comprising:
an outer sheath,
an inner thrombectomy catheter disposed inside of the outer sheath, the inner thrombectomy catheter including a distal end and a conveyer, the distal end having a distal tip being closed and a lateral opening such that the lateral opening receives a removed material, and the conveyer conveying the removed material toward a proximal end of the inner thrombectomy catheter, and
an expandable member disposed between the outer sheath and the inner thrombectomy catheter expanding radially when deployed from the outer sheath and contracting when retracted in the outer sheath; and
advancing the expandable member in a distal direction of the inner thrombectomy catheter such that the expandable member is deployed and expanded radially.

10. The method of claim 9, further comprising rotating the expandable member around an axis of the inner thrombectomy catheter.

11. The method of claim 9, further comprising applying aspiration forces through the lateral opening.

12. The method of claim 9, further comprising receiving the removed material through the lateral opening, and conveying the removed material toward the proximal end of the inner thrombectomy catheter by the conveyer.

13. The method of claim 9, further comprising retracting the expandable member in the outer sheath.

14. A surgical device, comprising:
a catheter assembly for removal of material from hollow bodies, comprising:
an outer sheath,
an inner thrombectomy catheter disposed inside of the outer sheath, the inner thrombectomy catheter including a distal end and a conveyer, the distal end having a distal tip and a lateral opening such that the lateral opening receives a removed material, and the conveyer conveying the removed material toward a proximal end of the inner thrombectomy catheter, and
an expandable member disposed between the outer sheath and the inner thrombectomy catheter expanding radially when deployed from the outer sheath and contracting when retracted in the outer sheath; and
a guidewire extendable through the inner thrombectomy catheter.

15. The surgical device of claim 14, wherein the expandable member is rotatable around an axis of the inner thrombectomy catheter independently of the inner thrombectomy catheter.

16. The surgical device of claim 15, further comprising a driving mechanism to rotate the expandable member.

17. The surgical device of claim 14, wherein the expandable member includes at least one prong that extends axially in a distal direction of the inner thrombectomy catheter.

18. The surgical device of claim 14, wherein the expandable member includes a plurality of prongs that extend axially in a distal direction of the inner thrombectomy catheter.

19. The surgical device of claim 14, wherein the distal end of the inner thrombectomy catheter and the expandable member are radially overlapped when the expandable member is deployed from the outer sheath while performing removal of the material.

20. The surgical device of claim 14, further comprising an aspiration device coupled to the inner thrombectomy catheter to create suction within the inner thrombectomy catheter.
